(19) United States
(12) Patent Application Publication
(10) Pub. No.: US 2007/0270477 A1
(43) Pub. Date: Nov. 22, 2007

(54) Agent for Prophylaxis or Treatment of Metabolic Syndrome

(75) Inventor: Nobuhiro Nishigaki, Osaka (JP)

Correspondence Address: FOLEY AND LARDNER LLP;SUITE 500, 3000 K STREET NW, WASHINGTON, DC, 20007, US

(73) Assignee: Takeda Pharmaceutical Company Limited

(21) Appl. No.: 11/664,107

(22) PCT Filed: Oct. 6, 2005
(86) PCT No.: PCT/JP05/18823

§ 371 Date: Mar. 29, 2007

(30) Foreign Application Priority Data
Oct. 7, 2004 (JP) 2004-294889
Apr. 22, 2005 (JP) 2005-125373

(51) Int. Cl. A61K 31/417; A61P 3/00

(52) U.S. Cl. 514/396

(57) ABSTRACT

The present invention provides an agent for the prophylaxis or treatment of metabolic syndrome, which comprises 2-ethoxy-1-[[2′-(5-oxo-2,5-dihydro-1,2,4-oxadiazol-3-yl)biphenyl-4-yl]methyl]-1H-benzimidazole-7-carboxylic acid or a salt thereof or a prodrug thereof and a PPAR agonist-like substance in combination.

### TECHNICAL FIELD

The present invention relates to an agent for the prophylaxis or treatment of metabolic syndrome.

### BACKGROUND ART

As a main cause of the cardiovascular diseases such as myocardial infarction and cerebral stroke that top the list of the causes of death in advanced countries, chronic diseases (what is called lifestyle-related diseases) such as diabetes, hyperlipidemia and hypertension have been considered. Recent epidemiological studies have reported that, when a multiplicity of these lifestyle-related diseases have been developed, the incidence of cardiovascular diseases is markedly high as compared to when a single lifestyle-related disease has been developed. Therefore, a new category so-called metabolic syndrome has been formed as a disease having a high risk (non-patent reference 1).

The metabolic syndrome refers to a pathology where the symptoms of obesity, hypertriglyceridemia, hypo-high-density-lipoproteinemia, impaired glucose tolerance, hypertension and the like are concurrently developed, and arteriosclerosis, a main cause of cardiovascular diseases, progress easily. To be specific, according to the criterion of WHO, patients with at least two of visceral obesity, dyslipidemia (high TG or low HDL) and hypertension in addition to hyperinsulinemia or impaired glucose tolerance are diagnosed as metabolic syndrome (World Health Organization: Definition, Diagnosis and Classification of Diabetes Mellitus and Its Complications. Part I: Diagnosis and Classification of Diabetes Mellitus, World Health Organization, Geneva, 1999). In addition, according to the criterion of Adult Treatment Panel III in National Cholesterol Education Program, that is an indicator for managing ischemic heart diseases in the United States, patients with at least three of visceral obesity, hypertriglyceridemia, low HDL cholesteremia, hypertension and impaired glucose tolerance are diagnosed as metabolic syndrome (National Cholesterol Education Program: Executive Summary of the Third Report of National Cholesterol Education Program (NCEP) Expert Panel on Detection, Evaluation, and Treatment of High Blood Cholesterol in Adults (Adults Treatment Panel III), non-patent reference 2).

Patent reference 1 describes benzimidazole derivatives having a potent angiotensin II antagonistic activity, which is useful as an agent for the prophylaxis or treatment of hypertension and the like. Patent reference 2 describes that, of the benzimidazole derivatives described in patent reference 1, a particular compound (2-ethoxy-1-[[2′-(5-oxo-2,5-dihydro-1,2,4-oxadiazol-3-yl)biphenyl-4-yl]methyl]-1H-benzimidazole-7-carboxylic acid: compound A) has an effect of improving insulin resistance in addition to an angiotensin II antagonistic activity, and is useful as an agent for the prophylaxis or treatment of diabetes and the like.

Patent reference 3 describes that a combination of a compound having an angiotensin II antagonistic activity and a compound having an effect of improving insulin resistance is useful for the prophylaxis or treatment of various angiotensin II-mediated diseases. Compounds having an effect of improving insulin resistance (e.g., troglitazone, pioglitazone, rosiglitazone etc.) have been employed clinically as highly superior therapeutic agents for diabetes and they are known to have a PPARγ agonistic activity (e.g., non-patent reference 3).

Patent reference 4 describes a combination of a compound having an angiotensin II antagonistic activity and a compound having an effect of improving insulin resistance.

Patent reference 5 generally describes that a compound having both a PPAR agonistic activity and an angiotensin II antagonistic activity is used for the prophylaxis or treatment of metabolic syndrome.

Patent reference 6 generally describes that a compound having a PPARα/γ agonistic activity and a compound having an angiotensin II antagonistic activity are used in combination to treat metabolic syndrome.

Patent reference 7 describes that a compound having an angiotensin II antagonistic activity suppresses body weight gain due to a PPARγ agonist-like substance.

patent reference 1: JP-A-5-271228

patent reference 2: JP-A-2003-231636

patent reference 3: JP-A-9-323940

patent reference 4: WO 2002/015933

patent reference 5: WO 2004/014308

patent reference 6: WO 2004/017896

patent reference 7: WO 2004/060399

non-patent reference 1: The Journal of the American Medical Association, Vol. 288, 2709-2716, 2002

non-patent reference 2: The Journal of the American Medical Association, Vol. 285, 2486-2497, 2001

non-patent reference 3: Journal of Pharmacology and Experimental Therapeutics, 284, 751-759, 1998

### DISCLOSURE OF THE INVENTION

Patients with metabolic syndrome have an extreme high incidence of cardiovascular diseases as compared to patients with a single lifestyle-related disease, the prophylaxis or treatment of metabolic syndrome is quite important to prevent cardiovascular diseases. While superior treatment methods of individual diseases such as diabetes, hyperlipidemia and hypertension have been established under the present situation, a pharmaceutical agent that exhibits a superior prophylactic or therapeutic effect for metabolic syndrome is desired, in view of the fact that one of the major final objects of the prophylaxis or treatment of lifestyle-related diseases is the prophylaxis of cardiovascular diseases.

The present inventors have found that, by combining a particular compound having an angiotensin II antagonistic activity with a compound having a PPARγ agonistic activity, a particularly remarkable prophylactic or therapeutic effect of the level higher than that achieved by adding the effects of the individual pharmaceutical agents can be exhibited on metabolic syndromes as well as advantages for a pharmaceutical agent in the safety, stability, dose, administration form, method of use and the like can be afforded, which resulted in the completion of the present invention.

Accordingly, the present invention relates to (1) An agent for the prophylaxis or treatment of metabolic syndrome, which comprises 2-ethoxy-1-[[2′-(5-oxo-2,5-dihydro-1,2,4-oxadiazol-3-yl)biphenyl-4-yl]methyl]-1H-benzimidazole-7-carboxylic acid or a salt thereof or a prodrug thereof and a PPARγ agonist-like substance in combination;
(2) the agent of the aforementioned (1), wherein the PPARγ agonist-like substance is pioglitazone or a salt thereof;
(3) a method for the prophylaxis or treatment of metabolic syndrome in a mammal, which comprises administering 2-ethoxy-1-[[2′-(5-oxo-2,5-dihydro-1,2,4-oxadiazol-3-yl)biphenyl-4-yl]methyl]-1H-benzimidazole-7-carboxylic acid or a salt thereof or a prodrug thereof and a PPARγ agonist-like substance to the mammal;
(4) use of 2-ethoxy-1-[[2′-(5-oxo-2,5-dihydro-1,2,4-oxadiazol-3-yl)biphenyl-4-yl]methyl]-1H-benzimidazole-7-carboxylic acid or a salt thereof or a prodrug thereof in combination with a PPARγ agonist-like substance for the production of an agent for the prophylaxis or treatment of metabolic syndrome; and the like.

### BRIEF DESCRIPTION OF DRAWINGS

FIG. 1 shows the effect of combined treatment of compound A (0.3 mg/kg) and pioglitazone hydrochloride (1 mg/kg) on systolic blood pressure in SHR/NDmcr-cp rats, wherein +/+; SHR/NDmcr-cp (+/+) rats, V; Vehicle-treated SHR/NDmcr-cp (fa/fa) rats, A; Compound A (0.3 mg/kg)-treated SHR/NDmcr-cp (fa/fa) rats, P; Pioglitazone hydrochloride (1 mg/kg)-treated SHR/NDmcr-cp (fa/fa) rats, A+P; (Compound A+Pioglitazone hydrochloride)-treated SHR/NDmcr-cp (fa/fa) rats. Data are presented as mean±SEM (n=5-8), *P<0.05, **P<0.01 vs. Vehicle-treated SHR/NDmcr-cp (fa/fa) rats (Dunnett's test).

FIG. 2 shows the effect of combined treatment of compound A (0.3 mg/kg) and pioglitazone hydrochloride (1 mg/kg) on plasma glucose, insulin, triglyceride and total cholesterol levels in SHR/NDmcr-cp rats, wherein +/+; SHR/NDmcr-cp (+/+) rats, V; Vehicle-treated SHR/NDmcr-cp (fa/fa) rats, A; Compound A (0.3 mg/kg)-treated SHR/NDmcr-cp (fa/fa) rats, P; Pioglitazone hydrochloride (1 mg/kg)-treated SHR/NDmcr-cp (fa/fa) rats, A+P; (Compound A+Pioglitazone hydrochloride)-treated SHR/NDmcr-cp (fa/fa) rats, data are presented as mean±SEM (n=5-8), *P<0.05, **P<0.01 vs. Vehicle-treated SHR/NDmcr-cp rats (Steel's test).

### BEST MODE FOR CARRYING OUT THE INVENTION

Since 5-oxo-2,5-dihydro-1,2,4-oxadiazol-3-yl group of Compound A to be used in the present invention has a tautomeric form, Compound A also refers to 2-ethoxy-1-[[2′-(5-oxo-4,5-dihydro-1,2,4-oxadiazol-3-yl)biphenyl-4-yl]methyl]-1H-benzimidazole-7-carboxylic acid.

Compound A to be used in the present invention can be produced by a method disclosed in patent reference 1 and the like.

Compound A to be used in the present invention may be itself or a pharmacologically acceptable salt. As such salt, a salt with an inorganic base (e.g., alkali metals such as sodium and potassium; alkaline earth metals such as calcium and magnesium; transition metal such as zinc, iron and copper; etc.); a salt with an organic base (e.g., organic amines such as trimethylamine, triethylamine, pyridine, picoline, tromethamine, ethanolamine, diethanolamine, triethanolamine, dicyclohexylamine, t-butylamine and N,N′-dibenzylethylenediamine; basic amino acids such as arginine, lysine and ornithine; etc.); and the like can be mentioned.

A prodrug of compound A or a salt thereof to be used in the present invention refers to a compound which is converted into compound A or a salt thereof by a reaction with an enzyme, stomach acid or the like under the physiological conditions in a living body, that is, a compound which undergoes enzymatic oxidation, reduction, hydrolysis or the like, and is changed into compound A or a salt thereof; a compound which undergoes hydrolysis by stomach acid or the like, and is changed into compound A or a salt thereof; or the like. For example, ester of compound A (e.g., methyl ester, ethyl ester, medoxomil ester etc.) can be used.

Compound A or a salt thereof or a prodrug thereof may be either a hydrate or a non-hydrate.

The PPARγ agonist-like substance to be used in the present invention may be any agonist of PPARγ, and may be any substance as long as it can exert a PPARγ agonistic activity.

The PPARγ agonist-like substance is, for example, preferably a substance exhibiting a clear in vitro PPARγ agonistic activity at a concentration of not more than 10 μM and the like.

As preferable examples of the PPARγ agonist-like substance, insulin sensitizers such as troglitazone, rosiglitazone, englitazone, ciglitazone, pioglitazone, PGJ₂, GI-262570, JTT-501, MCC-555, YM-440, KRP-297, CS-011 and FK-614 can be mentioned.

The PPARγ agonist-like substance includes a pharmacologically acceptable salt, and as such salt, a salt with an inorganic or organic base similar to those exemplified as the salt of compound A; a salt with an inorganic acid (e.g., hydrochloric acid, hydrobromic acid, nitric acid, sulfuric acid, phosphoric acid etc.); a salt with an organic acid (e.g., acetic acid, phthalic acid, fumaric acid, oxalic acid, tartaric acid, maleic acid, citric acid, succinic acid, methanesulfonic acid, p-toluenesulfonic acid etc.); and the like can be mentioned.

The PPARγ agonist-like substance to be used in the present invention may further have a PPARα functional regulatory activity (agonistic or antagonistic activity)

In the agent of the present invention, as a preferable combination of compound A or a salt thereof or a prodrug thereof and a PPARγ agonist-like substance, for example, a combination of compound A or a salt thereof and pioglitazone or a salt thereof (preferably pioglitazone hydrochloride) and the like can be mentioned.

An agent for the prophylaxis or treatment of metabolic syndrome of the present invention (herein sometimes to be simply abbreviated as “the agent of the present invention) has low toxicity and can be used for the prophylaxis and/or treatment of metabolic syndrome in mammals (e.g., human, mouse, rat, rabbit, dog, cat, bovine, horse, swine, monkey etc.).

Criteria for diagnosis of metabolic syndrome are announced by WHO in 1999, and by NCEP in 2001.

According to the criterion of WHO, patients with at least two of visceral obesity, dyslipidemia (high TG or low HDL) and hypertension in addition to hyperinsulinemia or impaired glucose tolerance are diagnosed as metabolic syndrome (World Health Organization: Definition, Diagnosis and Classification of Diabetes Mellitus and Its Complications. Part I: Diagnosis and Classification of Diabetes Mellitus, World Health Organization, Geneva, 1999). According to the criterion of Adult Treatment Panel III in National Cholesterol Education Program, that is an indicator for managing ischemic heart diseases in the United States, patients with at least three of visceral obesity, hypertriglyceridemia, low HDL cholesteremia, hypertension and impaired glucose tolerance are diagnosed as metabolic syndrome (National Cholesterol Education Program: Executive Summary of the Third Report of National Cholesterol Education Program (NCEP) Expert Panel on Detection, Evaluation, and Treatment of High Blood Cholesterol in Adults (Adults Treatment Panel III), non-patent reference 2).

In addition, according to the criterion of Japanese Ministry of Health, Labor and Welfare as regards benefits of worker's compensation insurance for the cost of secondary check in the regular health check, the cost of secondary check is paid when an individual has all of hypertension [systolic (maximum) blood pressure is not less than 140 mmHg or diastolic (minimum) blood pressure is not less than 90 mmHg], obesity [BMI is not less than 25], hyperglycemia [fasting blood glucose level is not less than 110 mg/dL, hemoglobin Alc is not less than 5.6%], and hyperlipidemia [total cholesterol is not less than 220 mg/dL or HDL cholesterol is less than 40 mg/dL or triglyceride (neutral fat) is not less than 150 mg/dL].

Moreover, according to the diagnostic criterion in Japan, metabolic syndrome is diagnosed when two or more items out of the following three items are met, with the basic conditions that the size of the circumference of the waist is not less than 85 cm for male and not less than 90 cm for female.
1) Hypertriglyceridemia (not less than 150 mg/dL) and/or Low HDL-cholesteremia (less than 40 mg/dL).
2) Systolic blood pressure is not less than 130 mmHg and/or diastolic blood pressure is not less than 85 mmHg.
3) Fasting blood glucose level is not less than 110 mg/dL.

In the present invention, the agent of the present invention encompasses either embodiment of administering compound A or a salt thereof or a prodrug thereof and PPARγ agonist-like substance as separate preparations or as a single combination preparation.

When a single combination preparation is to be prepared, compound A or a salt thereof or a prodrug thereof and PPARγ agonist-like substance are mixed as they are, or a pharmacologically acceptable carrier is further mixed by a method known per se to give a pharmaceutical composition.

When they are used in combination as separate preparations, the timing of administration of respective preparations is not particularly limited, and these may be administered to an administration subject simultaneously, or may be administered at different times. The dosage form of respective preparations may be the same or different, and as each dosage form, one generally employed for a pharmaceutical agent is appropriately selected depending on the active ingredient. Each preparation can be used in the form of each active ingredient as it is or a pharmaceutical composition prepared by mixing the active ingredient with a pharmacologically acceptable carrier according to a method known per se.

The content of compound A or a salt thereof or a prodrug thereof in the preparation is, for example, about 0.01 to about 99.9 wt %, preferably about 0.1 to about 99.9 wt %, more preferably about 0.5 to about 50 wt %, relative to a combination preparation (relative to each preparation when used in combination with individual preparations).

The content of a PPARγ agonist-like substance in the preparation is, for example, about 0.01 to about 99.9 wt %, preferably about 0.1 to about 99.9 wt %, more preferably about 0.5 to about 50 wt %, relative to a combination preparation (relative to each preparation when used in combination with individual preparations).

As a pharmaceutically acceptable carrier, various organic or inorganic carrier materials which are conventional as a pharmaceutical material are used, and is incorporated as an excipient, a lubricant, a binder or a disintegrant in a solid preparation; as a solvent, a solubilizer, a suspending agent, an isotonic agent, a buffer or a soothing agent in a liquid preparation. If necessary, pharmaceutical additives such as a preservative, an antioxidant, a colorant and a sweetener may be used.

The dosage form of the pharmaceutical composition includes oral preparations such as tablets, capsules (including soft capsules and microcapsules), granules, powders, syrups, emulsions, suspensions and sustained-releasing preparations; and parenteral preparations such as injections (e.g., subcutaneous injection, intravenous injection, intramuscular injection, intraperitoneal injection, intravitreous injection etc.), eye drops, external preparations (e.g., transnasal preparation, transdermal preparation, ointment etc.), suppositories (e.g., rectal suppository, vaginal suppository etc.), pellets and drops, and these can be safely administered either orally or parenterally.

The pharmaceutical compositions can be prepared by conventional methods in the technical field of pharmacy, such as the methods described in the Japanese Pharmacopoeia. Specific process for producing preparations will be described in detail below.

For example, oral preparations are produced by adding, for example, an excipient (e.g., lactose, sucrose, starch, D-mannitol etc.), a disintegrant (e.g., calcium carboxymethylcellulose etc.), a binder (e.g., gelatinized starch, gum arabic, carboxymethylcellulose, hydroxypropylcellulose, polyvinylpyrrolidone etc.) or a lubricant (e.g., talc, magnesium stearate, polyethylene glycol 6000 etc.) to an active ingredient, compression-molding them and, if necessary, coating the molded material with a coating base (e.g., a sugar-coated base, a water-soluble film coating base, an enteric film coating base, a sustained-releasing film coating base etc.) by a method known per se for the purpose of taste masking, enteric coating or sustainability.

Injections are prepared by dissolving, suspending or emulsifying an active ingredient together with a dispersing agent (e.g., polysorbate 80, polyoxyethylene hydrogenated castor oil 60, polyethylene glycol, carboxymethylcellulose, sodium arginate etc.), a preservative (e.g., methylparaben, propylparaben, benzyl alcohol, chlorobutanol, phenol etc.), an isotonic (e.g., sodium chloride, glycerin, D-mannitol, D-sorbitol, glucose etc.) in an aqueous solvent (e.g., distilled water, physiological saline, Ringer's solution etc.) or an oily solvent (e.g., vegetable oil such as olive oil, sesame oil, cottonseed and corn oil, propylene glycol etc.). If desired, additives such as a solubilizer (e.g., sodium salicylate, sodium acetate etc.), a stabilizer (e.g., human serum albumin etc.) and a soothing agent (benzyl alcohol etc.) may be used.

The daily dose and administration ratio of each active ingredient in the agent of the present invention can be appropriately selected according to the administration subject, administration route, target disease, condition, combination of active ingredients and the like, and each active ingredient only needs to be not more than the maximum dose when it is used solely for clinical application.

For example, while the dose of compound A (dose as compound A when administered as a salt of compound A, a prodrug of compound A, or a prodrug of a salt of compound A) for oral administration to an adult patient with metabolic syndrome (body weight 60 kg) varies depending on the administration subject, administration route, target disease, condition and the like, it is about 0.001 to about 500 mg, preferably about 0.1 to about 100 mg, more preferably about 1 to about 60 mg as a daily dose. These amounts may be administered once a day, or in 2 or 3 portions.

As a dose of a PPARγ agonist-like substance (e.g., pioglitazone hydrochloride), the daily dose is about 0.1 to about 600 mg, preferably about 0.5 to about 240 mg, more preferably about 1.0 to about 100 mg. These amounts may be administered once a day, or in 2 or 3 portions.

The administration ratio of compound A and a PPARγ agonist-like substance (PPARγ agonist-like substance/compound A) is about 0.0002 to about 600000, preferably about 0.005 to about 2400, more preferably about 0.02 to about 100, further preferably about 0.1 to about 50.

The mammal to be the application subject for the present invention may be any of animals having environmental factor causality including diet and the like or a genetic background, or animals having both factors of environmental factor and a genetic background. For example, these animals may have developed hyperglycemia, hypertension and the like attributable to obesity, or may have developed hypertension and hyperlipidemia attributable to hyperglycemia and obesity, and the order of onset is not particularly limited.

Moreover, the agent of the present invention improves blood pressure and plural plasma parameters (e.g., triglyceride level, glucose level and the like) of mammals affected with metabolic syndrome and can be used for treating patients with lifestyle-related diseases, who are affected with insulin resistance, impaired glucose tolerance; diabetes (e.g., noninsulin dependent diabetes, type II diabetes, type II diabetes associated with insulin resistance, type II diabetes associated with impaired glucose tolerance etc.); hyperinsulinemia; various complications such as hypertension associated with insulin resistance, hypertension associated with impaired glucose tolerance, hypertension associated with diabetes (e.g., type II diabetes etc.), hypertension associated with hyperinsulinemia, hypertension associated with hypertriglyceridemia, hypertension associated with low HDL-cholesteremia, insulin resistance occurring in association with hypertension, impaired glucose tolerance occurring in association with hypertension, diabetes occurring in association with hypertension, hyperinsulinemia occurring in association with hypertension, diabetic complications [e.g., microangiopathy, diabetic neuropathy, diabetic nephropathy, diabetic retinopathy, diabetic cataract, large vessel disease, osteopenia, diabetic hyperosmolar coma, infectious diseases (e.g., respiratory infectious disease, urinary tract infectious disease, digestive infectious disease, infectious disease of dermal soft tissue, infectious disease of inferior limb etc.), diabetic gangrene, dry mouth, lowered sense of hearing, diabetic cerebrovascular disorder, diabetic impairment of peripheral blood flow, diabetic hypertension etc.], diabetic cachexia and diabetic nephropathy.

The combination of compound A or a salt thereof or a prodrug thereof with a PPARγ agonist-like substance can be also used for the prophylaxis or treatment of the target disease of a compound having an angiotensin II antagonistic activity. As the target disease of a compound having an angiotensin II antagonistic activity, diseases developed or whose onset is promoted by contraction and growth of blood vessels or organ disorders that express via angiotensin II receptor, by the presence of angiotensin II, or by the factors induced by the presence of angiotensin II, and the like can be mentioned.

As such diseases, for example, hypertension, blood pressure circadian rhythm abnormality, heart diseases (e.g., cardiac hypertrophy, acute heart failure and chronic heart failure including congestive heart failure, cardiac myopathy, angina pectoris, myocarditis, arrhythmia, tachycardia, cardiac infarction etc.), cerebrovascular disorders (e.g., asymptomatic cerebrovascular disorder, transient ischemic attack, cerebral stroke, cerebrovascular dementia, hypertensive encephalopathy, cerebral infarction etc.), cerebral edema, cerebral circulatory disorder, recurrence and sequela of cerebrovascular disorders (e.g., neurotic symptom, psychic symptom, subjective symptom, disorder in daily living activities etc.), ischemic peripheral circulation disorder, myocardial ischemia, venous insufficiency, progression of cardiac insufficiency after cardiac infarction, renal diseases (e.g., nephritis, glomerulonephritis, glomerulosclerosis, renal failure, thrombotic microvasculopathy, complication of dialysis, organ dysfunction including nephropathy due to irradiation etc.), arteriosclerosis including atherosclerosis (e.g., aneurysm, coronary arteriosclerosis, cerebral arteriosclerosis, peripheral arteriosclerosis etc.), vascular hypertrophy, vascular hypertrophy or obliteration and organ disorders after intervention (e.g., percutaneous transluminal coronary angioplasty, stenting, coronary angioscopy, intravascular ultrasound, intracoronary thrombolytic therapy etc.), vascular re-obliteration and restenosis after bypass, polycythemia, hypertension, organ disorder and vascular hypertrophy after transplantation, rejection after transplantation, ocular diseases (e.g., cataract, diabetic retinopathy, glaucoma, ocular hypertension etc.), thrombosis, multiple organ failure, endothelial dysfunction, hypertensive tinnitus, sleep apnea syndrome, migraine, other circulatory diseases (e.g., deep vein thrombosis, obstructive peripheral circulatory disorder, arteriosclerosis obliterans, thromboangiitis obliterans, ischemic cerebral circulatory disorder, Raynaud's disease, Buerger's disease etc.), metabolic or nutritional disorders (e.g., obesity, hyperlipidemia, hypercholesterolemia, hyperuratemia, hyperkalemia, hypernatremia etc.), neurodegenerative diseases (e.g., Alzheimer's disease, Parkinson's syndrome, amyotrophic lateral sclerosis, AIDS encephalopathy etc.), central nervous system disorders (e.g., disorders such as cerebral hemorrhage and cerebral infarction, and their sequela and complication, head injury, spinal injury, cerebral edema, sensory malfunction, sensory dysfunction, autonomic nervous system malfunction, autonomic nervous system dysfunction, multiple sclerosis etc.), dementia, defects of memory, consciousness disorder, amnesia, anxiety symptom, catatonic symptom, discomfort mental state, psychoses (e.g., depression, epilepsy, alcoholism etc.), inflammatory diseases (e.g., arthritis such as rheumatoid arthritis, osteoarthritis, rheumatoid myelitis, periostitis etc.; inflammation after operation or injury; remission of swelling; pharyngitis; cystitis; pneumonia; atopic dermatitis; inflammatory intestinal diseases such as Crohn's disease, ulcerative colitis etc.; meningitis; inflammatory ocular disease; inflammatory pulmonary disease such as pneumonia, pulmonary silicosis, pulmonary sarcoidosis, pulmonary tuberculosis etc.), allergic diseases (e.g., allergic rhinitis, conjunctivitis, gastrointestinal allergy, pollinosis, anaphylaxis etc.), chronic obstructive pulmonary disease, interstitial pneumonia, pneumocystis carinni pneumonia, collagen diseases (e.g., systemic lupus erythematodes, scleroderma, polyarteritis etc.), hepatic diseases (e.g., hepatitis including chronic hepatitis, hepatic cirrhosis etc.), portal hypertension, digestive system disorders (e.g., gastritis, gastric ulcer, gastric cancer, gastric disorder after operation, dyspepsia, esophageal ulcer, pancreatitis, colon polyp, cholelithiasis, hemorrhoidal disease, varices ruptures of esophagus and stomach etc.), blood or myelopoietic diseases (e.g., erythrocytosis, vascular purpura, autoimmune hemolytic anemia, disseminated intravascular coagulation syndrome, multiple myelopathy etc.), bone diseases (e.g., fracture, refracture, osteoporosis, osteomalacia, bone Paget's disease, sclerosing myelitis, rheumatoid arthritis, osteoarthritis of the knee and joint tissue destruction due to similar diseases etc.), solid tumor, tumors (e.g., malignant melanoma, malignant lymphoma, cancer of digestive organs (e.g., stomach, intestine etc.) etc.), cancer and cachexia following cancer, metastasis of cancer, endocrinopathy (e.g., Addison's disease, Cushing's syndrome, pheochromocytoma, primary aldosteronism etc.), Creutzfeldt-Jakob disease, urinary organ or male genital diseases (e.g., cystitis, prostatic hypertrophy, prostatic cancer, sex infectious disease etc.), female disorders (e.g., climacteric disorder, gestosis, endometriosis, hysteromyoma, ovarian disease, mammary gland disease, sex infectious disease etc.), diseases relating to environment or occupational factor (e.g., radiation hazard, hazard by ultraviolet, infrared, or laser beam, altitude sickness etc.), respiratory diseases (e.g., cold syndrome, pneumonia, asthma, pulmonary hypertension, pulmonary thrombosis, pulmonary embolism etc.), infectious diseases (e.g., viral infectious diseases with cytomegalovirus, influenza virus, herpes virus etc., rickettsiosis, bacterial infectious disease etc.), toxemias (e.g., sepsis, septic shock, endotoxin shock, Gram-negative sepsis, toxic shock syndrome etc.), otorhinolaryngological diseases (e.g., Meniere's syndrome, tinnitus, dysgeusia, vertigo, disequilibrium, dysphagia etc.), skin diseases (e.g., keloid, hemangioma, psoriasis etc.), intradialytic hypotension, myasthenia gravis, systemic diseases such as chronic fatigue syndrome can be mentioned.

In addition, long-term suppression of action of angiotensin II results in the improvement or suppression of promotion of disorder or abnormality in the biofunction and physiological action, that causes adult disorders and various diseases associated with aging and the like, which in turn leads to the primary and secondary prophylaxis of diseases or clinical conditions caused thereby or suppression of the progression thereof. As the disorder or abnormality in the biofunction and physiological action, for example, disorder or abnormality in automatic controlling capability of cerebral circulation or renal circulation, disorder of circulation (e.g., peripheral circulation, cerebral circulation, microcirculation etc.), disorder of blood-brain-barrier, salt sensitivity, abnormal state of coagulation and fibrinolysis system, abnormal state of blood and blood cell components (e.g., enhancement of platelet aggregation, malfunction of erythrocyte deformability, enhancement of leukocyte adhesiveness, rise of blood viscosity etc.), enhancement of production and function of growth factor or cytokines (e.g., PDGF, VEGF, FGF, interleukin, TNF-α, MCP-1 etc.), enhancement of production and infiltration of inflammatory cells, enhancement of production of free radical, enhancement of lipomatosis, endothelial function disorder, dysfunctions of endothelium, cell and organ, edema, morphogenesis of cells such as smooth muscle (morphogenesis to proliferation type etc.), enhancement of production and function of vasoactive substance or thrombosis inducers (endothelin, thromboxane A₂ etc.), abnormal constriction of blood vessel etc., metabolic disorder (serum lipid abnormalities, dysglycemia etc.), abnormal growth of cell etc., angiogenesis (including abnormal vasculogenesis during abnormal capillary network formation in adventitia of atherosclerotic lesion) and the like can be mentioned. Of these, the present invention can be used as an agent for the primary and/or secondary prophylaxis or treatment of organ disorders associated with various diseases (e.g., cerebrovascular disorder and organ disorder associated therewith, organ disorder associated with circulatory disease, organ disorder associated with diabetes, organ disorder after intervention etc.) can be mentioned.

The combination of compound A or a salt thereof or a prodrug thereof with a PPARγ agonist-like substance can be also used for the prophylaxis or treatment of the target disease of a PPARγ agonist-like substance. The applicable diseases of the PPARγ agonist-like substance include, for example, diabetes (e.g., type I diabetes, type II diabetes, gestational diabetes mellitus and the like), hyperlipidemia (e.g., hypetriglyceridemia, hypercholesterolemia, hypo-high density lipoproteinemia, postprandial hyperlipidemia and the like), diabetic complications (e.g., neuropathy, nephropathy, retinopathy, cataract, macroangiopathy, osteopenia and the like), impaired glucose tolerance (IGT), obesity, osteoporosis, cachexia (e.g., carcinomatous cachexia, tuberculous cachexia, diabetic cachexia, cachexia due to hemopathy, cachexia due to endocrinopathy, cachexia due to infection, or cachexia due to acquired immune deficiency syndrome), fatty liver, hypertension, polycystic ovary syndrome, gestational diabetes mellitus, renal diseases (e.g., diabetic nephropathy, glomerulonephritis, glomerulosclerosis, nephrotic syndrome, hypertensive nephrosclerosis, end stage renal disease and the like), muscular dystrophy, myocardial infarction, angina pectoris, cerebrovascular disorders (e.g., cerebral infarction, cerebral stroke), insulin resistance syndrome, hyperinsulinemia, sensory disturbance in hyperinsulinemia, tumors (e.g., leukemia, breast cancer, prostatic cancer, skin carcinoma and the like), irritable bowel syndrome, acute or chronic diarrhea, visceral obesity syndrome and the like. In addition, the PPARγ agonist-like substance can be used for the treatments aiming at improved insulin resistance, enhanced insulin sensitivity, and suppression of the shift from impaired glucose tolerance to diabetes.

Furthermore, the agent of the present invention can be used in combination with pharmaceutical agents such as a therapeutic agent for diabetes, a therapeutic agent for diabetic complications, an anti-hyperlipidemia agent, an anti-hypertensive agent, an anti-obesity agent, a diuretic, a chemotherapeutic agent, an immunotherapeutic agent, and the like (hereinafter to be abbreviated as a combination drug). Moreover, the agent of the present invention can be also used in combination with vaccine preparations such as angiotensin vaccine, or gene treatment for peripheral arterial obstruction etc., or regenerative medicines using embryonic stem cells, or the like. When the agent of the present invention is used in combination with a combination drug, the agent of the present invention and the combination drug may be administered as independent pharmaceutical agents or a combined preparation as a single pharmaceutical agent.

When used in combination as independent pharmaceutical agents, the timing of administration of the agent of the present invention and that of the combination drug are not limited. They may be administered simultaneously or at staggered times to the administration subject. Furthermore, two or more kinds of combination drugs may be used in combination at an appropriate ratio.

The dose of the combination drug can be suitably determined based on the dose clinically employed for each agent. In addition, the administering ratio of the agent of the present invention and a combination drug can be determined depending on the administration subject, administration route, subject disease, the symptom, combination and the like.

As the therapeutic agent for diabetes, for example, insulin preparations (e.g., animal insulin preparations extracted from the bovine or swine pancreas; human insulin preparations synthesized by a genetic engineering technique using E. coli or a yeast, and the like), α-glucosidase inhibitors (e.g., voglibose, acarbose, miglitol, emiglitate etc.), biguanides (e.g., phenformin, metformin, buformin etc.), insulin secretagogues [e.g., sulfonylureas (e.g., tolbutamide, glibenclamide, gliclazide, chlorpropamide, tolazamide, acetohexamide, glyclopyramide, glimepiride, glipizide, glybuzole etc.), repaglinide, senaglinide, nateglinide, mitiglinide or its calcium salt hydrate, GLP-1 etc.], amyrin agonists (e.g., pramlintide etc.), phosphotyrosine phosphatase inhibitors (e.g., vanadic acid etc.), can be mentioned.

As the therapeutic agents for diabetic complications, for example, aldose reductase inhibitors (e.g., tolrestat, epalrestat, zenarestat, zopolrestat, minalrestat, fidarestat, SNK-860, CT-112 etc.), neurotrophic factors (e.g., NGF, NT-3, BDNF etc.), neurotrophic factor production-promoting agents, PKC inhibitors (e.g., LY-333531 etc.), AGE inhibitors (e.g., ALT946, pimagedine, pyratoxathine, N-phenacylthiazolium bromide (ALT766), EXO-226 etc.), active oxygen scavengers (e.g., thioctic acid etc.), cerebral vasodilators (e.g., tiapride, mexiletine etc.) and the like can be mentioned.

As the anti-hyperlipidemia agents, for example, statin compounds which are cholesterol synthesis inhibitors (e.g., pravastatin, simvastatin, lovastatin, atorvastatin, fluvastatin, cerivastatin, itavastatin or a salt thereof (e.g., sodium salt etc.) etc.), squalene synthetase inhibitors or fibrate compounds having a triglyceride lowering effect (e.g., bezafibrate, clofibrate, simfibrate, clinofibrate etc.) and the like can be mentioned.

As the antihypertensive agents, for example, angiotensin converting enzyme inhibitors (e.g., captopril, enalapril, delapril, quinapril etc.), angiotensin II antagonists (e.g., losartan, candesartan, candesartan cilexetil, eprosartan, valsantan, termisartan, irbesartan, tasosartan, olmesartan, olmesartan medoxomil etc.), calcium antagonists (e.g., manidipine, nifedipine, amlodipine, efonidipine, nicardipine, azelnidipine, clinidipine etc.) and the like can be mentioned.

As the anti-obesity agents, for example, central acting anti-obesity agent (e.g., dexfenfluramine, fenfluramine, phentermine, sibutramine, amfepramone, dexamphetamine, mazindol, phenylpropanolamine, clobenzorex, rimonabant etc.), pancreatic lipase inhibitors (e.g., orlistat etc.), β3 agonist (e.g., CL-316243, SR-58611-A, UL-TG-307, SB-226552, AJ-9677, BMS-196085, AZ40140 etc.), anorectic peptides (e.g., leptin, CNTF (ciliary neurotropic factor) etc.), cholecystokinin agonists (e.g., lintitript, FPL-15849 etc.) and the like can be mentioned.

As the diuretics, for example, xanthine derivatives (e.g., theobromine sodium salicylate, theobromine calcium salicylate etc.), thiazide preparations (e.g., ethiazide, cyclopenthiazide, trichlormethiazide, hydrochlorothiazide, hydroflumethiazide, benzylhydrochlorothiazide, penfluthiazide, polythiazide, methyclothiazide etc.), anti-aldosterone preparations (e.g., spironolactone, triamterene, eplerenone etc.), carbonic anhydrase inhibitors (e.g., acetazolamide etc.), chlorobenzenesulfonamide preparations (e.g., chlortalidone, mefruside, indapamide etc.), azosemide, isosorbide, ethacrynic acid, piretanide, bumetanide, furosemide and the like can be mentioned.

As the chemotherapeutic agents, for example, alkylating agents (e.g., cyclophosphamide, ifosphamide etc.), metabolic antagonists (e.g., methotrexate, 5-fluorouracil etc.), anticancer antibiotics (e.g., mitomycin, adriamycin etc.), plant-derived anticancer agents (e.g., vincristine, vindesine, taxol etc.), cisplatin, carboplatin, etoposide and the like can be mentioned. Of these, furtulon, neofurtulon etc., which are 5-fluorouracil derivatives, and the like are preferable.

As the immunotherapeutic agents, for example, microorganism or bacterial components (e.g., muramyl dipeptide derivative, picibanil etc.), polysaccharides having immunostimulatory activity (e.g., lenthinan, schizophyllan, krestin etc.), cytokines obtained by genetic engineering techniques (e.g., interferon, interleukin (IL) etc.), colony stimulating factor (e.g., granulocyte-colony stimulating factor, erythropoietin etc.) and the like can be mentioned, with preference given to IL-1, IL-2, IL-12 and the like.

Moreover, pharmaceutical agents having a cachexia improving effect acknowledged in animal models and clinical situations, which include cyclooxygenase inhibitors (e.g., indomethacin etc.), progesterone derivatives (e.g., megestrol acetate), glucosteroid (e.g., dexamethasone etc.), metoclopramide pharmaceutical agents, tetrahydrocannabinol pharmaceutical agents, fat metabolism improving agents (e.g., eicosapentaenoic acid etc.), growth hormone, IGF-1, and antibodies against TNF-α, LIF, IL-6 and oncostatin M, which is a factor inducing cachexia, and the like, can be also used in combination with the agent of the present invention.

When the agent of the present invention is used in combination with a combination drug, the amounts of these agents can be decreased in a safe range in consideration of opposition effect of these agents. Therefore, the side effects expected to be induced by the combination of these agents can be safely prevented. In addition, the dose of the combination drug can be reduced, and as a result, the side effects expected to be induced by the combination drug can be effectively prevented.

The combination drug used in combination with the agent of the present invention preferably includes biguanides (e.g., phenformin, metformin, buformin etc.); sulfonylureas (e.g., tolbutamide, glibenclamide, gliclazide, chlorpropamide, tolazamide, acetohexamide, glyclopyramide, glimepiride, glipizide, glybuzole etc.); statin compounds (e.g., pravastatin, simvastatin, lovastatin, atorvastatin, fluvastatin, cerivastatin, itavastatin or a salt thereof (e.g., sodium salt etc.) etc.); squalene synthetase inhibitors; fibrates (e.g., bezafibrate, clofibrate, simfibrate, clinofibrate etc.); calcium antagonists (e.g., manidipine, nifedipine, amlodipine, efonidipine, nicardipine, azelnidipine, clinidipine etc.); Single-pill amlodipine besylate/atorvastatin calcium (e.g. Caduet); central acting anti-obesity agent (e.g., dexfenfluramine, fenfluramine, phentermine, sibutramine, amfepramone, dexamphetamine, mazindol, phenylpropanolamine, clobenzorex, rimonabant etc.); pancreatic lipase inhibitors (e.g., orlistat etc.); and the like.

The agent of the present invention exhibits a superior effect in the prophylaxis or treatment of metabolic syndrome. While patients with metabolic syndrome show a high incidence of onset of cardiovascular diseases, the agent of the present invention can suppress the onset of cardiovascular diseases by prophylaxis or treatment of thereof, reduce the severity of the diseases and strikingly improve the QOL (Quality of Life) of the patients.

The present invention is explained in detail in the following by referring to Experimental Examples and Examples. However, these Examples do not limit the present invention.

As the ingredients (additives) other than the active ingredient in the formulations described as Examples, those listed in the Japanese Pharmacopoeia, Japanese Pharmaceutical Codex or Japanese Pharmaceutical Excipients can be used.

### TEST EXAMPLE 1

Effect of concomitant administration of 2-ethoxy-1-{[2′-(5-oxo-2,5-dihydro-1,2,4-oxadiazol-3-yl)biphenyl-4-yl]methyl}-1H-benzimidazole-7-carboxylate (compound A) and pioglitazone hydrochloride on rat with metabolic syndrome

1) Test Method

As a metabolic syndrome model animal, male SHR/NDmcr-cp (fa/fa) rats (obtained from Disease Model Cooperative Research Association) were used. Blood was drawn from the tail vein of 15-week-old rats, and the levels of glucose, triglyceride and insulin in plasma were measured. As SHR/NDmcr-cp (fa/fa) rats reveal hypercholesterolemia in addition to metabolic syndrome, plasma total cholesterol level was also measured. Insulin level was quantified by radioimmunoassay (Shionolia Insulin, Shionogi). Systolic blood pressure was measured by tail-cuff method (Softron BP-98A, Softron) with incubation in a chamber at 37° C. SHR/NDmcr-cp (fa/fa) rats were divided into 4 groups (9 per group) to make the above-mentioned parameters equal, and five SHR/NDmcr-cp (+/+) rats were used as control rats. Compound A (0.3 mg/kg) and pioglitazone hydrochloride (1 mg/kg) were orally administered independently or in combination once a day on consecutive days from the time point of 16 weeks of age (0.5% methylcellulose solution was administered to a vehicle group). The blood pressure was measured at 12 weeks after the administration (5 hr after administration), blood was drawn from the tail vein at 13 weeks after administration, and the levels of glucose, triglyceride, insulin and total cholesterol in plasma were determined. The values are all expressed in mean±standard error (SEM). The statistical analysis was conducted by Dunnett's test using the values of the vehicle administration group and the drug administration group in the case of homogeneity of variance, and in the case of heterogeneity of variance, Steel's test was used.

2) Results

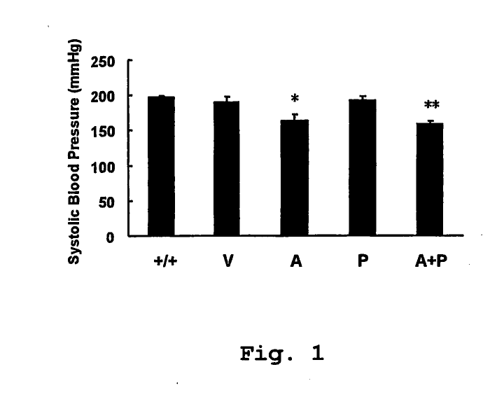

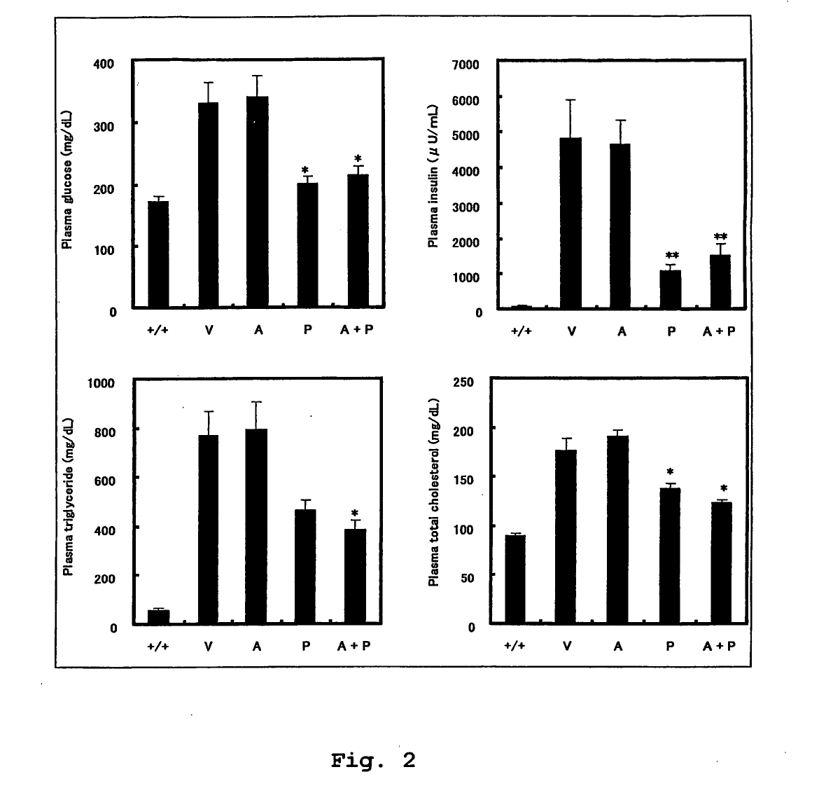

Results are shown in FIGS. 1 and 2. The pathology of metabolic syndrome was exhibited by SHR/NDmcr-cp (fa/fa), which maintained high blood-pressure almost equivalent to that of control SHR/NDmcr-cp (+/+) rats, and showed increase in the plasma glucose, triglyceride, insulin and total cholesterol levels.

Only the concomitant administration group of compound A and pioglitazone hydrochloride showed significant decrease in all of blood pressure, plasma glucose, triglyceride, insulin and total cholesterol exhibiting a remarkable improvement in the metabolic syndrome index.

EXAMPLE 1
Capsule

| | | | |
|---|---|---|---|
| (1) | Compound A | 5 | mg |
| (2) | Pioglitazone hydrochloride | 30 | mg |
| (3) | Lactose | 85 | mg |
| (4) | Microcrystalline cellulose | 70 | mg |
| (5) | Magnesium stearate | 10 | mg |
| | 1 capsule | 200 | mg |

(1), (2), (3), (4) and ½ of (5) are admixed and granulated. Thereto is added the remaining (5), and the total amount is sealed in a gelatin capsule.

EXAMPLE 2
Tablet

| | | | |
|---|---|---|---|
| (1) | Compound A | 10 | mg |
| (2) | Pioglitazone hydrochloride | 30 | mg |
| (3) | Lactose | 35 | mg |
| (4) | Corn starch | 140 | mg |
| (5) | Microcrystalline cellulose | 30 | mg |
| (6) | Magnesium stearate | 5 | mg |
| | 1 tablet | 250 | mg |

(1), (2), (3), (4), ⅔ of (5) and ½ of (6) are admixed and granulated. Thereto are added the remaining (5) and (6), and the mixture is compression formed to give tablets.

### Industrial Applicability

The agent of the present invention has a superior effect on metabolic syndrome. While patients with metabolic syndrome show a high incidence of onset of cardiovascular diseases, the agent of the present invention can suppress the onset of cardiovascular diseases by prophylaxis or treatment thereof, reduce the severity of the diseases and strikingly improve QOL of the patients.

1. An agent for the prophylaxis or treatment of metabolic syndrome, which comprises 2-ethoxy-1-[[2′-(5-oxo-2,5-dihydro-1,2,4-oxadiazol-3-yl)biphenyl-4-yl]methyl]-1H-benzimidazole-7-carboxylic acid or a salt thereof or a prodrug thereof and a PPARγ agonist-like substance in combination.

2. The agent according to claim 1, wherein the PPARγ agonist-like substance is pioglitazone or a salt thereof.

3. A method for the prophylaxis or treatment of metabolic syndrome in a mammal, which comprises administering 2-ethoxy-1-[[2′-(5-oxo-2,5-dihydro-1,2,4-oxadiazol-3-yl)biphenyl-4-yl]methyl]-1H-benzimidazole-7-carboxylic acid or a salt thereof or a prodrug thereof and a PPARγ agonist-like substance to the mammal.

4. (canceled)
